(19) United States
(12) Patent Application Publication
(10) Pub. No.: US 2011/0288420 A1
(43) Pub. Date: Nov. 24, 2011

(54) BLOOD PRESSURE MEASURING DEVICE AND BLOOD PRESSURE MEASURING METHOD

(75) Inventors: Tomonori MANO, Matsumoto (JP); Toshihiko YOKOYAMA, Matsumoto (JP)

(73) Assignee: SEIKO EPSON CORPORATION, Tokyo (JP)

(21) Appl. No.: 13/101,716

(22) Filed: May 5, 2011

(30) Foreign Application Priority Data
May 19, 2010 (JP) 2010-115524

(51) Int. Cl. A61B 5/021

(52) U.S. Cl. 600/485

(57) ABSTRACT

A blood pressure measuring device includes a blood flow velocity sensor detecting a blood flow within the body; a blood flow velocity sensor driver driving the blood flow velocity sensor part; a blood flow velocity sensor signal calculating part controlling the blood flow velocity sensor driver and the blood flow velocity sensor, and obtaining the blood flow velocity within the body; a vascular diameter sensor detecting a difference in reflection arrival time for the vascular wall in the body; a vascular diameter sensor driver driving the vascular diameter sensor; a vascular diameter sensor signal calculating part controlling the vascular diameter sensor driver and the vascular diameter sensor, and obtaining the vascular diameter in the body; and a blood pressure signal calculater using a result of a calculation by the blood flow velocity sensor signal calculater and the vascular diameter sensor signal calculater to obtain the blood pressure of the subject.

### CROSS-REFERENCE TO RELATED APPLICATIONS

This application claims priority to Japanese Patent Application No. 2010-115044 filed on May 19, 2010. The entire disclosure of Japanese Patent Application No. 2010-115044 is hereby incorporated herein by reference.

### BACKGROUND

1. Technological Field

The invention relates to a blood pressure measuring device and a blood pressure measuring method.

2. Background Technology

Methods for using ultrasound to measure blood pressure are currently being proposed as methods for measuring blood pressure. For example, the maximum diameter and the minimum diameter are obtained in relation to a localized portion of an artery, these parameter values are entered into a non-linear function, and the non-linear function is used to convert the diameter at each input time, whereby the pressure at each of the times at the localized portion is calculated (e.g., see Patent Citation 1.)

There has also been proposed a method for using ultrasound to detect the blood flow velocity, flow rate, volume, or another parameter; using light waves to detect the pulse wave velocity; relating the first parameter to the pulse wave velocity; and calculating the blood pressure and variation thereof (e.g., see Patent Citations 2 and 3.)

JP-A 2004-041382 (Patent Citation 1), JP-A 4-250135, and JP-A 2004-154231 (Patent Citation 3) are examples of the related art.

### SUMMARY

### Problems to be Solved by the Invention

However, conventional calculations of the blood pressure value using ultrasound such as those described in Patent Citations 1 through 3 require calibration using a cuff-type blood pressure monitor. This presents an inconvenience in that, in order to perform 24-hour ambulatory blood pressure monitoring (“24h ABPM”) or to continuously measure the blood pressure for each beat, the cuff must remain attached to the body or be carried around for use at appropriate times; and presents a risk of being impractical for use in normal life.

Also, in addition to the necessity of calibration using a cuff-type blood pressure monitor, there is also a risk of a further problem being presented in that the calibration is required at a regular interval (approximately 30 minutes to 1 hour). Increasing the interval between calibrations is generally known to increase the error probability when the blood pressure value is estimated from the pulse wave velocity. This is because although vascular elasticity characteristics (E0: vascular elasticity rate under zero pressure, γ: constant for a specific blood vessel) can be regarded as being constant within a short duration of time, the error becomes greater when the time exceeds a certain length. In Patent Citation 1, a stiffness parameter β is calculated from a maximum blood pressure Ps and a minimum blood pressure Pd obtained using the cuff-type blood pressure monitor. Since there is a correlation between the stiffness parameter β and the vascular elasticity described above, it shall be apparent that the value varies when the time exceeds a certain length. In other words, in order to obtain an accurate blood pressure value in a continuous and continual manner, it is not sufficient merely to perform the calibration once; it is necessary for the calibration to be performed at a certain interval, e.g., about once an hour.

### Means Used to Solve the Above-Mentioned Problems

The invention has been contrived in order to solve at least some of the above-mentioned problems, and can be achieved through one of the following embodiments or application examples.

### First Application Example

A blood pressure measuring device including: a blood flow velocity sensor part for transmitting/receiving a wave motion relative to a body surface of a subject to blood within the body, and detecting a blood flow within the body; a blood flow velocity sensor driving part for driving the blood flow velocity sensor part; a blood flow velocity sensor signal calculating part for controlling the blood flow velocity sensor driving part and the blood flow velocity sensor part, and obtaining the blood flow velocity within the body; a vascular diameter sensor part for transmitting/receiving ultrasound with respect to a blood vessel in the body, and detecting a difference in reflection arrival time for the vascular wall in the body; a vascular diameter sensor driving part for driving the vascular diameter sensor part; a vascular diameter sensor signal calculating part for controlling the vascular diameter sensor driving part and the vascular diameter sensor part, and obtaining the vascular diameter in the body; and a blood pressure signal calculating part for using a result of a calculation by the blood flow velocity sensor signal calculating part and the vascular diameter sensor signal calculating part to obtain a blood pressure of the subject.

According to the application example described above, it is possible to provide a blood pressure measuring device that can be continuously worn, in which a correction coefficient is initially obtained using a blood pressure value measured using a cuff-type blood pressure monitor, whereby it is subsequently possible to measure the blood pressure to a high degree of accuracy without using a cuff-type blood pressure monitor; and in an instance in which ambulatory blood pressure monitoring is performed by the subject, calibration can be performed in a simple manner without using a cuff-type blood pressure monitor.

### Second Application Example

The blood pressure measuring device described above, wherein the blood pressure signal calculating part performs a calculation to obtain the blood pressure by converting the vascular diameter to a head pressure.

According to the application example described above, the vascular diameter and the blood pressure can be regarded as undergoing a substantially linear change, therefore making it possible to measure the temporal change in the vascular diameter to obtain a value that correlates with the temporal change in the blood pressure.

### Third Application Example

The blood pressure measuring device described above further includeing a height position sensor part for obtaining a difference in the height of a predetermined portion of the subject between a first state and a second state, the first state being a state in which the predetermined portion of the subject is positioned at a predetermined height, and a second state being a state in which the predetermined portion is positioned at a height of the heart of the subject; wherein the head pressure is obtained using the height difference measured using the height position sensor part.

According to the application example described above, it is possible to readily measure the height difference, which is one of the elements in terms of obtaining the head pressure.

### Fourth Application Example

The blood pressure measuring device described above, wherein the blood flow velocity sensor part includes a transmitting element and a receiving element; the transmitting element and the receiving element are provided in a plurality of pairs; and an angle between a direction of propagation of the transmitted/received wave motion and the direction of blood flow differs between each of the pairs.

According to the application example described above, it is possible to obtain the blood flow velocity, even in an instance in which the angle between the blood vessel and the wave motion is unknown.

### Fifth Application Example

The blood pressure measuring device described above, wherein the blood flow velocity sensor part is made using a piezoelectric element.

According to the application example described above, it is possible to decrease the size of the blood flow velocity sensor since a piezoelectric element has a simple structure.

### Sixth Application Example

A method for measuring a blood pressure of a subject in a first state in which a predetermined portion of the subject is positioned at a predetermined height, the blood pressure being proportional, by a predetermined constant of proportionality, to a value obtained by dividing the blood flow velocity at the predetermined portion by the square of the vascular diameter at the predetermined portion, the blood pressure measuring method characterized in including obtaining the constant of proportionality; measuring the vascular diameter and the blood flow velocity at the predetermined portion in the first state; obtaining the blood pressure using the vascular diameter, the blood flow velocity, and the constant of proportionality; displaying the blood pressure; and determining whether calibration of the constant of proportionality is necessary.

According to the application example described above, it is possible to provide a blood pressure measuring method that can be continuously worn, in which a correction coefficient is initially obtained using a blood pressure value measured using a cuff-type blood pressure monitor, whereby it is subsequently possible to measure the blood pressure to a high degree of accuracy without using a cuff-type blood pressure monitor; and in an instance in which ambulatory blood pressure monitoring is performed by the subject, calibration can be performed in a simple manner without using a cuff-type blood pressure monitor.

### Seventh Application Example

The blood pressure measuring method described above, wherein the obtaining the constant of the proportionality includes: measuring, in a second state in which the predetermined portion is positioned at a height of the heart of the subject, each of the vascular diameter of the predetermined portion and the vascular diameter during a systolic phase and a diastolic phase of the predetermined portion, and obtaining a first average vascular diameter, an average systolic-phase vascular diameter, and an average diastolic-phase vascular diameter; for measuring, in the first state, a difference in the height of the predetermined portion between the first state and the second state; using the height difference to obtain the head pressure between the first state and the second state; measuring, in the first state, each of the vascular diameter of the predetermined portion and the blood flow velocity and the vascular diameter during the systolic phase and the diastolic phase of the predetermined portion, and obtaining a second average vascular diameter, a systolic-phase blood flow velocity, a systolic-phase vascular diameter, a diastolic-phase blood flow velocity, and a diastolic-phase vascular diameter; using the first average vascular diameter and the second average vascular diameter to obtain a change in the average vascular diameter; using the head pressure, the change in the average vascular diameter, the average systolic-phase vascular diameter, and the average diastolic-phase vascular diameter to obtain a blood pressure difference between a systolic-phase blood pressure and a diastolic-phase blood pressure; and using the blood pressure difference, the systolic-phase blood flow velocity, the systolic-phase vascular diameter, the diastolic-phase blood flow velocity, and the diastolic-phase vascular diameter to obtain the constant of proportionality.

According to the application example described above, it is possible to readily calibrate the constant of proportionality.

### Eighth Application Example

The blood pressure measuring method described above, wherein the measuring, in the first state, the difference in the height of the predetermined portion includes performing a measurement using a height position sensor part for measuring a difference in the height of the predetermined portion between the first state and the second state.

According to the application example described above, it is possible to readily measure the height difference, which is one of the elements in terms of obtaining the head pressure.

### BRIEF DESCRIPTION OF THE DRAWINGS

FIG. 1 is an external view showing a state in which a blood pressure measuring device according to a present embodiment is being worn;

FIG. 2 is a view showing a blood flow velocity sensor and a vascular diameter sensor according to the present embodiment;

FIG. 3 is a view showing a circuit block according to the present embodiment;

FIG. 4 is a view showing measurement positions of the blood pressure measuring device according to the present embodiment;

FIG. 5 is a view showing a vascular diameter when a head pressure component is applied according to the present embodiment;

FIG. 6 is a diagram showing a relationship between the vascular wall pressure and the vascular diameter (volume) according to the present embodiment;

FIG. 7 is a diagram showing cuff pressurization measurement values according to the present embodiment;

FIG. 8 is a view showing a blood flow velocity sensor according to the present embodiment;

FIG. 9 is a diagram showing a measurement method according to the present embodiment; and

FIG. 10 is a diagram showing a calibration routine according to the present embodiment.

### DETAILED DESCRIPTION OF EXEMPLARY EMBODIMENTS

A description of the present embodiment will now be described with reference to the accompanying drawings. The drawings used are displayed in an expanded or a contracted form as appropriate so that the portion to be described is in a readily recognized state.

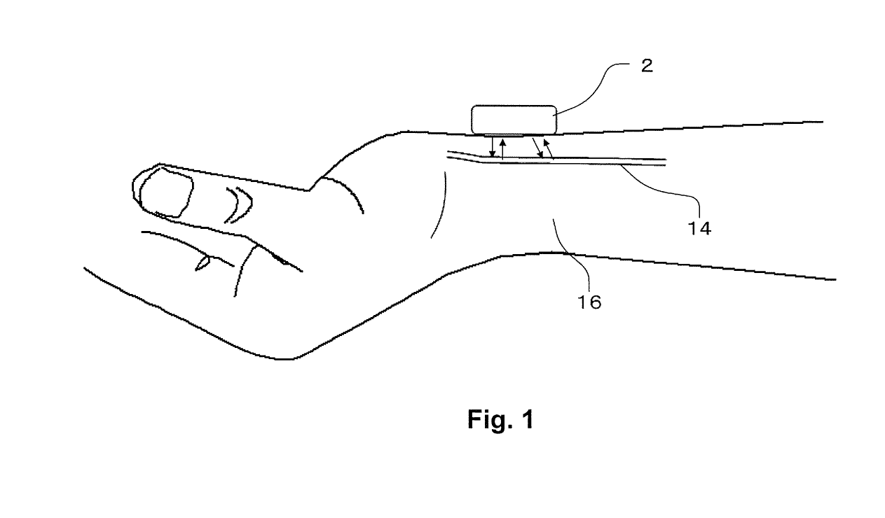

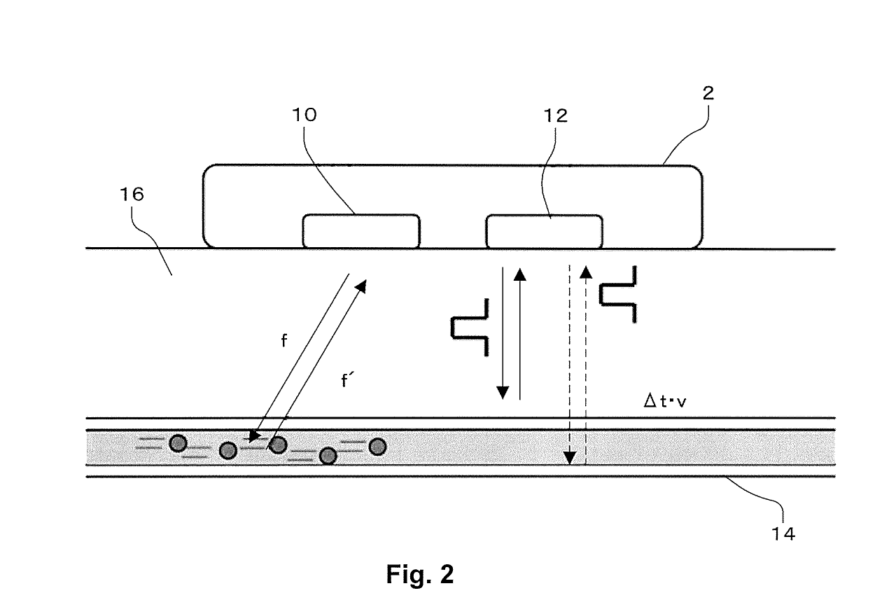

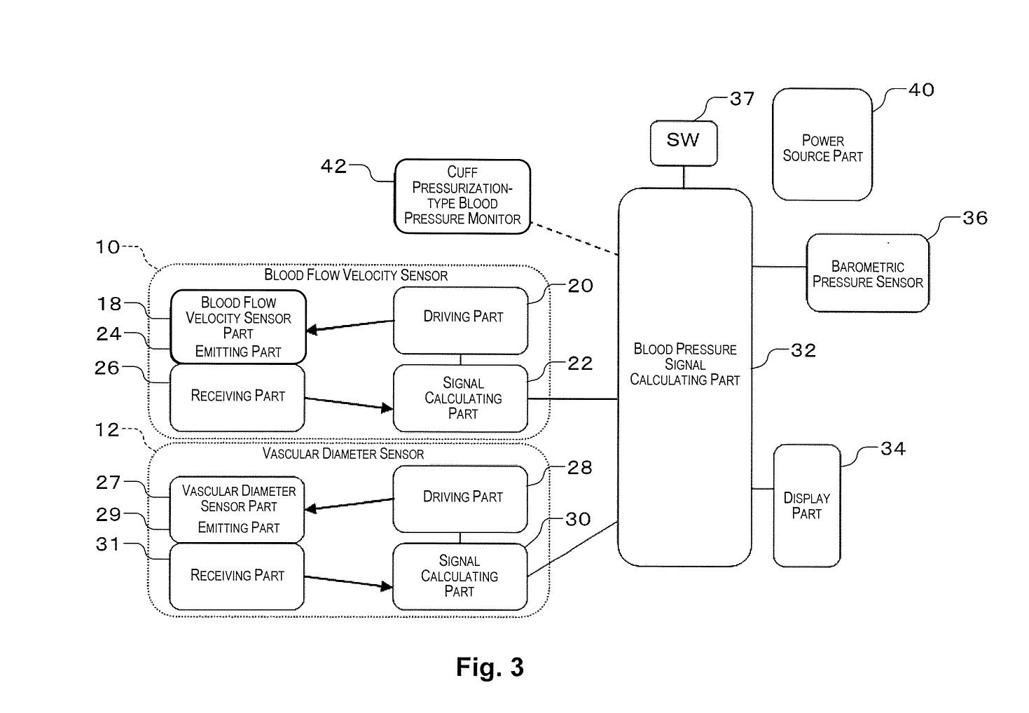

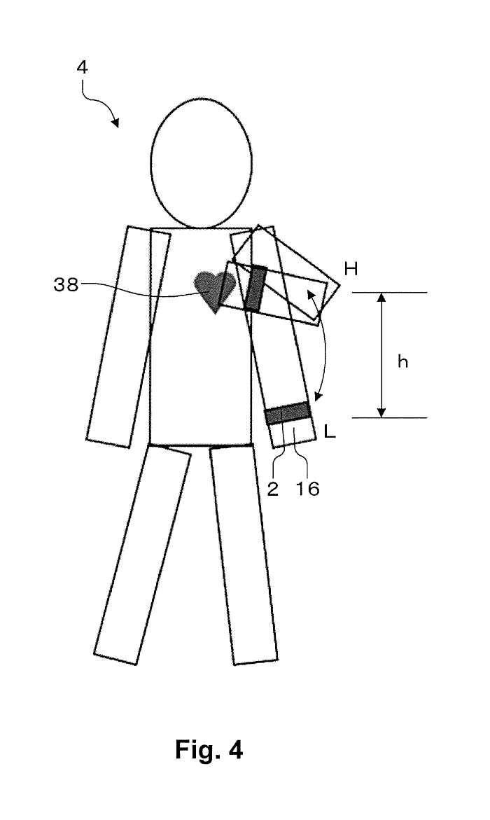

FIG. 1 is an external view showing a state in which a blood pressure measuring device according to a present embodiment is being worn. FIG. 2 is a view showing a blood flow velocity sensor and a vascular diameter sensor according to the present embodiment. FIG. 3 is a view showing a circuit block according to the present embodiment. The blood pressure measuring device 2 according to the present embodiment includes a blood flow velocity sensor 10 and a vascular diameter sensor 12. The blood pressure measuring device 2 is worn on a wrist section 16 of a subject 4 (see FIG. 4). The blood pressure measuring device 2 measures the blood flow velocity v and the vascular diameter d of a radial artery (i.e., a blood vessel) 14, and obtains a blood pressure P.

The blood flow velocity sensor 10 is mounted at a position so as to enable emission of ultrasound at the radial artery 14 inside the wrist section 16. The blood flow velocity sensor 10 performs mixing of a basic wave motion f, emitted from the blood flow velocity sensor 10, and a received wave motion f. A blood flow velocity sensor signal calculating part (i.e., a signal calculating part) 22 detects the mixed wave motion, and thereby extracts only the frequency component that corresponds with a Doppler shift. The signal calculating part 22 calculates the blood flow velocity v from this Doppler frequency component Δf (=f−f′) and an angle θ between the wave motions f, f′ and the radial artery 14.

The blood flow velocity sensor 10 includes a blood flow velocity sensor part 18, a blood flow velocity sensor driving part (i.e., a driving part) 20, and a signal calculating part 22. The blood flow velocity sensor part 18 transmits/receives a wave motion relative to a body surface of the subject 4 to blood within the body, and detects the blood flow within the body. The blood flow velocity sensor part 18 includes an emitting part (i.e., a transmitting element) 24 and a receiving part (i.e., a receiving element) 26. The emitting part 24 and the receiving part 26 are provided in a plurality of pairs, and the angle between a direction of propagation of the transmitted/received wave motion and the radial artery 14 differs between each of the pairs. The driving part 20 drives the blood flow velocity sensor part 18. The signal calculating part 22 controls the driving part 20 and the blood flow velocity sensor part 18, and obtains the blood flow velocity v within the body. The blood flow velocity sensor part 18 is made using a piezoelectric element. It is thereby possible to decrease the size of the blood flow velocity sensor since a piezoelectric element has a simple structure.

The vascular diameter sensor 12 is mounted at a position so as to enable emission of ultrasound at the radial artery 14 inside the wrist section 16. The vascular diameter sensor 12 transmits a burst signal or a pulse signal having a frequency of several megahertz to several tens of megahertz, and measures, from the transmitted wave and the received wave, the time taken for a wave reflected at the radial artery 14 to arrive. A vascular diameter sensor part 27 transmits/receives ultrasound with respect to the radial artery 14 within the body and detects a difference in reflection arrival time for the wall of the radial artery 14 within the body.

The vascular diameter sensor 12 includes the vascular diameter sensor part 27, a vascular diameter sensor driving part (i.e., a driving part) 28, and a vascular diameter sensor signal calculating part (i.e., a signal calculating part) 30. The vascular diameter sensor part 27 includes an emitting part 29 and a receiving part 31. The vascular diameter sensor part 27 transmits/receives ultrasound with respect to the radial artery 14 within the body, and detects a difference in reflection arrival time for the wall of the radial artery 14 within the body. The driving part 28 drives the vascular diameter sensor part 27. The signal calculating part 30 controls the driving part 28 and the vascular diameter sensor part 27, and obtains the vascular diameter d within the body.

The blood pressure measuring device 2 according to the present embodiment includes a blood pressure signal calculating part 32, a display part 34, an barometric pressure sensor (i.e., a height position sensor part) 36, a switch 37, and a power source part 40. The blood pressure signal calculating part 32 uses the result of the calculations by the signal calculating part 22 and the signal calculating part 30 to obtain the blood pressure P of the subject 4. The display part 34 displays the blood pressure P of the subject 4. The blood pressure P may also be displayed as a graph or otherwise visualized. The pulse rate may also be similarly displayed. The display part 34 also displays a message to indicate that calibration is required. The barometric pressure sensor 36 measures the height position of the blood pressure measuring device 2. The switch 37 switches between supply and disconnection of electrical power from the power source part 40 to each of the functional parts of the blood pressure measuring device 2. The power source part 40 feeds power to each of the functional parts of the blood pressure measuring device 2. In the present embodiment, it is assumed that the power source part 40 is, e.g., a rechargeable secondary battery.

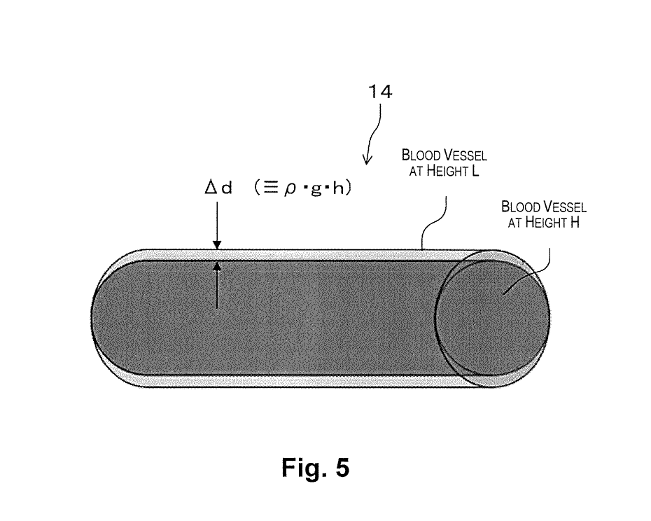

FIG. 4 is a view showing measurement positions of the blood pressure measuring device 2 according to the present embodiment. FIG. 5 is a view showing the vascular diameter d when a head pressure component is applied according to the present embodiment. A description will now be given for a non-invasive blood pressure measurement method for measuring the blood flow velocity v and the vascular diameter d without using a cuff (i.e., a compression garment) and calculating the blood pressure P. The blood pressure P is obtained from a product of the blood flow rate Q and the vascular resistance R.

P=Q·R (1)

Here, the blood flow rate Q is obtained from a product of the vascular diameter d and the blood flow velocity v as shown in Formula (2).

Q=(π·d2·v)/8 (2)

Also, the vascular resistance R is determined by the ratio of the viscosity η of the blood flowing through the radial artery 14 and the vascular diameter d, wherein there exists a relationship in which the vascular resistance R decreases with increasing vascular diameter d. If C is treated as a constant.

R=η·C/d4 (3)

When blood pressure P is derived taking these relational expressions into account, a change in the strength of the volume pulse wave known as the pulse wave is actually a change in the vascular diameter d when the blood undergoes pulsation being captured as a change in volume. Measuring the volume pulse wave makes it possible to measure a value that correlates with the vascular diameter d, and to measure a value that correlates with the vascular resistance R. Measuring the blood flow velocity v in the blood vessel makes it possible to also obtain a value that correlates with the blood flow rate Q, and therefore makes it possible to measure the blood pressure P.

A description will now be given for calculation of a systolic-phase blood pressure Psys and the diastolic-phase blood pressure Pdia can be obtained as shown in Formulas (4) and (5), using Formulas (1) through (3).

Psys=π/8·η·C·vsys/dsys2 (4)

Pdia=π/8·η·C·vdia/ddia2 (5)

Thus, the blood pressure difference between the systolic-phase blood pressure Psys and the diastolic-phase blood pressure Pdia can be obtained as shown in Formula (6).

Psys−Pdia=π/8η·C·(vsys/dsys2−vdia/ddia2) (6)

Here, vsys is the systolic-phase blood flow velocity, dsys is the systolic-phase vascular diameter, vdia is the diastolic-phase blood flow velocity, and ddia is the diastolic-phase vascular diameter.

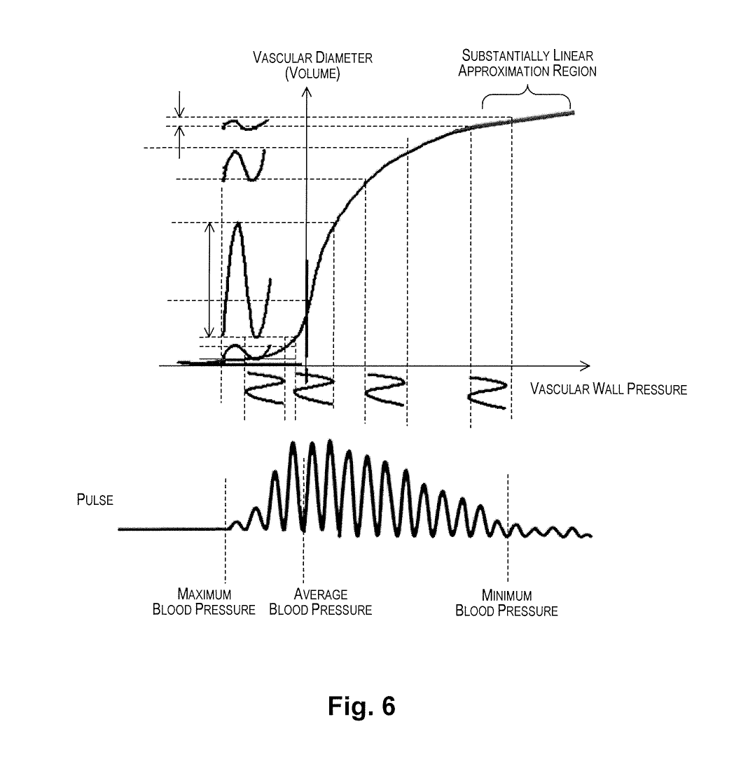

FIG. 6 is a diagram showing a relationship between the vascular wall pressure and the vascular diameter (volume) according to the present embodiment. FIG. 6 is a diagram showing a tube law of the blood vessel. In a conventional blood pressure measurement using cuff pressurization, a non-linear region of the tube law is used in order to obtain an oscillometric waveform. In contrast, in the present embodiment, a substantially linear approximation region shown in FIG. 6 is used. In this portion, the vascular diameter d and the vascular wall pressure (i.e., the blood pressure P) can be regarded as changing in a substantially linear fashion. Therefore, measuring the temporal change in the vascular diameter d makes it possible to obtain a value that correlates with the temporal change in the blood pressure P.

A description will now be given for a method for using the above formulae to calculate the systolic-phase blood pressure Psys and the diastolic-phase blood pressure Pdia. First, the systolic-phase blood flow velocity vsys, the systolic-phase vascular diameter dsys, the diastolic-phase blood flow velocity vdia, and the diastolic-phase vascular diameter ddia are obtained at a height H that is identical to the position of the heart 38, i.e., in a state in which the head pressure does not require correction. A wave motion is transmitted/received with respect to the blood vessel within the body, the systolic-phase blood flow velocity vsys and the diastolic-phase blood flow velocity vdia are calculated from the amount of Doppler shifting of the wave scattered by the blood flow, and the systolic-phase vascular diameter dsys and the diastolic-phase vascular diameter ddia are calculated from the difference in reflection arrival time for the two vascular walls. At the same time, the temporal change in the vascular diameter d is measured. Using the tube law of the blood vessel, the vascular diameter d and the vascular wall pressure (i.e., the blood pressure P) can be approximated to a substantially linear form when no pressure or very little pressure is being applied. Here, the temporal change in the vascular diameter d is similar to the temporal change in the blood pressure P (see FIG. 6).

Next, the vascular diameter d is similarly measured at a position L, which is lower than the position of the heart 38 by height h. In this instance, if the subject 4 is in a stable state, the blood vessel is subjected to an additional pressure corresponding to a head pressure component compared to the position of the heart 38. Specifically, measuring the temporal change in the vascular diameter d again in this state makes it possible to obtain the temporal change in the blood pressure P that includes the additional head pressure component (see FIG. 5). It is thereby possible to obtain a change Δd in the vascular diameter d corresponding with the head pressure (ρ·g·h; where ρ is the blood density and g is gravitational acceleration). The change in the vascular diameter d during the systolic phase and the diastolic phase can be obtained by measurement, and the blood pressure difference ΔP (=Psys−Pdia) between the systolic-phase blood pressure Psys and the diastolic-phase blood pressure Pdia can also be calculated. Substituting this value in Formula (6) allows the constant of proportionality (π/8·η·C) to be obtained. Therefore, the actual systolic-phase blood pressure Prsys and the actual diastolic-phase blood pressure Prdia can be calculated from Formulae (4) and (5).

The blood density ρ is about 1.055±0.005 g/cm² depending on the individual, and the effect on the blood pressure value therefore varies between individuals by several tenths of a millimeter of mercury. Therefore, the blood density ρ can be regarded as being constant. It can be seen that a correct value for the head pressure (ρ·g·h) can be obtained if the height is measured accurately. According to the present embodiment, calibration using a cuff-type blood pressure monitor or another blood pressure monitor is not necessary, and the head pressure can be used to perform calibration in an extremely simple manner. Also, it is not necessary to measure the volume pulse wave; constant monitoring of the blood pressure can be performed merely by using wave motion to measure the blood flow velocity and the vascular diameter.

Method for Converting the Head Pressure (ρ·g·h) into Vascular Diameter d

In a state in which the blood pressure measuring device according to the present embodiment is worn on the wrist section 16 as shown in FIG. 4, measurement of the temporal change in the vascular diameter d, and measurement of the actual systolic-phase blood pressure Prsys and the actual diastolic-phase blood pressure Prdia using a cuff pressurization-type blood pressure monitor 42, are performed at a position at height H which is equal to the height of the heart 38. Next, the arm is lowered to a position at height L, and the temporal change in the vascular diameter d is measured. It is thereby possible to calculate the amount of change in the vascular diameter d that corresponds with the pressure value resulting from the head pressure (see FIG. 5.)

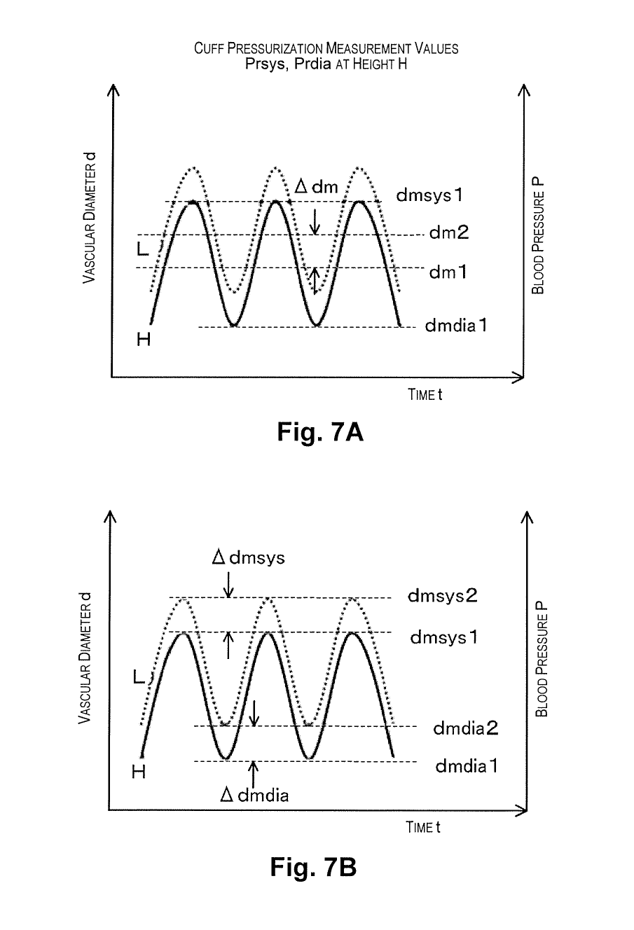

FIG. 7 is a diagram showing scuff pressurization measurement values according to the present embodiment. Calculation of the amount of change in the vascular diameter d that corresponds with the pressure value resulting from the head pressure can be performed in one of the following methods (a) through (c).

(a) The change in the vascular diameter d is measured for approximately 10 seconds, and the average vascular diameter at the position of each of heights H and L (i.e., dm1 and dm2) in FIG. 4 is measured. Then, the change Δdm in the average vascular diameter (dm1, dm2) is obtained using Formula (7).

Δdm=dm2−dm1 (7)

The change Δd in the vascular diameter that corresponds to the head pressure is obtained using Formula (8).

Δd=Δdm (8)

Using the average systolic-phase vascular diameter dmsys1 and the average diastolic-phase vascular diameter dmdia1 at height H in FIG. 4, Formula (9) is thereby satisfied with regards to the relationship between pressure and vascular diameter.

(Prsys−Prdia):ρ·g·h=(dmsys1−dmdia1):Δdm (9)

The head pressure (ρ·g·h) is thereby obtained from Formula (10) (see FIG. 7A.)

(ρ·g·h)=(Prsys−Prdia)·Δdm/(dmsys1·dmdia1) (10)

(b) The change in the vascular diameter d is measured for approximately 10 seconds, and the average systolic-phase vascular diameter (dmsys1, dmsys2) and the average diastolic-phase vascular diameter (dmdia1, dmdia2) at the position of each of heights H and L in FIG. 4 are measured. Then, the change (Δdmsys) in the average systolic-phase vascular diameter (dmsys1 and dmsys2) is obtained using Formulae (11), and the change (Δdmdia) in the average diastolic-phase vascular diameter (dmdia1, dmdia2) is obtained using Formulae in (12).

Δdmsys=dmsys2−dmsys1 (11)

Δdmdia=dmdia2−dmdia1 (12)

Taking the average of the above, the change Δd in the vascular diameter that corresponds to the head pressure is obtained using Formula (13).

Δd=(Δdmsys+Δdmdia)/2 (13)

Formula 14 is thereby satisfied with regards to the relationship between pressure and vascular diameter.

(Prsys−Prdia):ρ·g·h=(dmsys1−dmdia1):(Δdmsys+Δdmdia)/2 (14)

The head pressure (ρ·g·h) is thereby obtained from Formula (15) (see FIG. 7B.)

ρ·g·h=(Prsys−Prdia)·(Δdmsys+Δdmdia)/2·(dmsys1−dmdia1) (15)

(c) In the above methods (a) and (b), calculation was performed based on the concept of using the substantially linear approximation region shown in FIG. 6. However, a more accurate method of measurement will now be described. First, the temporal change in the vascular diameter d at the position at height H in FIG. 4 is used to calculate the temporal change in the vascular volume V. In general, the relationship between the vascular volume V and the pressure difference Pt between the intravascular pressure and the cuff pressure can be represented by Formula (16). Therefore, using a value of b=0.03 mmHg−1, V0 and Vmax can be obtained from the relationship for the vascular volume (Vrsys, Vrdia) at actual systolic-phase blood pressure Prsys and actual diastolic-phase blood pressure Prdia. The temporal change in the pressure difference Pt between the intravascular pressure and the cuff pressure at the position at height H can thereby be calculated from the temporal change in the vascular volume V.

V=Vmax+(V0−Vmax)·eb·Pt (16)

Next, the temporal change in the vascular diameter d at the position at height L is used to calculate the temporal change in the vascular volume (Vrsys, Vrdia), and Formula (16) is used to obtain the temporal change in the pressure difference Pt between the intravascular pressure and the cuff pressure. The temporal change in the pressure difference Pt between the intravascular pressure and the cuff pressure at each of the positions at heights H and L is used to obtain the difference in the average value of the pressure difference Pt between the intravascular pressure and the cuff pressure with respect to each of the positions, and the obtained value is used as the head pressure (ρ·g·h). Alternatively, the difference between the average systolic-phase blood pressure and the average diastolic-phase blood pressure at each of the positions is obtained, and the average value of the differences is used as the head pressure. If conversion of the head pressure (ρ·g·h) and the vascular diameter d (vascular volume) can be performed, the blood pressure difference (Prsys−Prdia) between the actual systolic-phase blood pressure Prsys and the actual diastolic-phase blood pressure Prdia can be obtained as shown in Formula (17).

Prsys−Prdia=1/b·log [(Vsys−Vmax)/(Vdia−Vmax)] (17)

Here, Vsys is the systolic-phase vascular volume, and Vdia is the diastolic-phase vascular volume.

If the head pressure (ρ·g·h) can be calculated, the blood pressure difference (Prsys−Prdia) between the actual systolic-phase blood pressure Prsys and the actual diastolic-phase blood pressure Prdia can be obtained merely by measuring the vascular diameter d, using the relationships described above. Calculating the head pressure (ρ·g·h) once before the start of continuous monitoring, such as at the start of the day, makes it possible to improve the accuracy of measurement. Also, the height difference h between the height H and the height L of the measurement positions is a crucial parameter that affects accuracy; therefore, each measurement is performed at the same position. For example, the height H is defined as the position of the heart 38, the height L is defined as the position at which the arm has been directly lowered, and the height difference h is measured. Alternatively, a high-precision barometric pressure sensor 36 or a similar device may be used to calculate the height. It is thereby possible to readily measure the height difference, which is one of the elements in terms of obtaining the head pressure.

### Method for Measuring Vascular Diameter

When the vascular diameter d is measured, the driving part 28 of the vascular diameter sensor 12 shown in FIG. 3 is used to transmit a burst signal or a pulse signal having a frequency of several megahertz to several tens of megahertz as shown in FIG. 2, and to measure, from the transmitted wave and the wave received by the receiving part 26, the time taken for the wave reflected at the vascular wall to arrive. If, for example, the time taken for the reflected wave to arrive is 1.73 μs and the speed of sound within the body is 1500 m/s, then it is possible to calculate the vascular diameter d as 2.6 mm. For example, a piezoelectric element may be used for transmission/reception of ultrasound. A known method for measuring the vascular diameter d is the echo tracking method, in which the vascular wall or another tissue is tracked based on an echo signal obtained from an ultrasound beam. The echo tracking method makes it possible to measure the displacement of the vascular wall or another tissue to an accuracy of several microns, equal to or less than the wavelength of the ultrasound.

### Method for Measuring Blood Flow Velocity

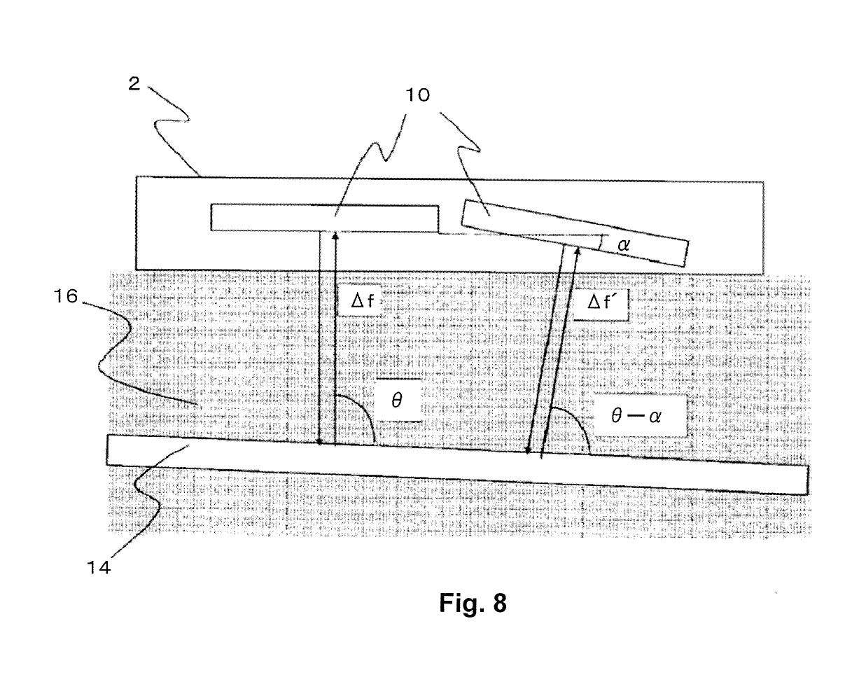

FIG. 8 is a view showing a blood flow velocity sensor according to the present embodiment. When the blood flow velocity v is measured, the basic wave motion f emitted from the driving part 20 of the blood flow velocity sensor 10 shown in FIG. 3, and the wave motion f received at the receiving part 26 (see FIG. 2) are mixed. The signal calculating part 22 detects the mixed wave motion, and thereby extracts only the frequency component that corresponds with a Doppler shift. The signal calculating part 22 calculates the blood flow velocity from this Doppler frequency component Δf (=f−f′) and the angle θ between the wave motion and the radial artery 14 using Formula (18).

v=ε·Δf/(2·f·cos θ) (18)

Here, ε represents the speed of sound within the body, f represents the frequency of the inputted wave motion, v is the blood flow velocity, and θ is the angle between the radial artery 14 and the wave motion. In reality, it is difficult to determine the angle θ between the wave motion and the radial artery 14. Therefore, two blood flow velocity sensors are used, the sensors being capable of transmitting/receiving two ultrasound wave motion having an angle θ and θ−α respectively in relation to the direction of the blood flow to be measured, so that a plurality of blood flow velocity sensors such as those shown in FIG. 8 can be used to obtain the blood flow velocity v even when the angle θ between the wave motion and the radial artery 14 is unknown. When α represents the angle between the two blood flow velocity sensors, it is possible to obtain the angle θ between the wave motion and the radial artery 14. Specifically, the blood flow velocity sensor for transmitting/receiving the wave motion with respect to the body surface to within the body is provided in a pair. When α represents the angle between the Doppler frequency components Δf0 and Δf1 received by the respective blood flow velocity sensors, and between the two blood flow velocity sensors, 0 is obtained using Formula (19).

θ=tan⁻¹(Δf1/Δf0−cos α)/sin α (19)

The angle θ between the wave motion and the radial artery 14 obtained here is substituted into Formula (18), and the Doppler frequency component Δf0 is substituted into Formula (18) as Δf=Δf0, whereby the blood flow velocity v is obtained.

For example, in order to obtain the blood flow velocity v, a pulse signal of 1 MHz is transmitted, and the Doppler frequency component Δf of the received wave is calculated. If the Doppler frequency component Δf is 0.33 kHz and the angle θ between the radial artery 14 and the wave motion is 45°, the blood flow velocity v can be calculated as about 50 cm/s. The vascular diameter d and the blood flow velocity v obtained as described above are used to calculate the blood pressure P for each beat. Specifically, the vascular diameter d and the blood flow velocity v are measured for every beat using ultrasound or another wave motion as shown in Formulae (4) and (5), and the blood pressure P is established. The constant of proportionality (π/8·η·C) in Formulae (4) and (5) is obtained from Formula (20), which is a modification of Formula (6).

π/8·η·C=(Psys−Pdia)/(vsys/dsys2−vdia/ddia2) (20)

The blood pressure P is thereby calculated, using the relationships shown in Formulae (4) and (5), at a given sampling rate or a regular interval, thereby making it possible to perform constant blood pressure monitoring in a stable manner without pressurization.

### Simple Calibration Method

Since biological information is reflected in the constant of proportionality (π/8·η·C) to a considerable extent, it is necessary to perform calibration of the value at a certain interval. In such an instance, the vascular diameter d and the blood flow velocity v at each of the positions at height H and height L as shown in FIG. 4 are obtained using ultrasound or another wave motion as described above, and the blood pressure difference (Psys−Pdia) between the systolic-phase blood pressure Psys and the diastolic-phase blood pressure Pdia is obtained using conversion of the head pressure (ρ·g·h) and the vascular diameter d. Calibration can thereby be performed at appropriate time even without cuff pressurization.

### Blood Pressure Measuring Method and Calculation of Calibration Value

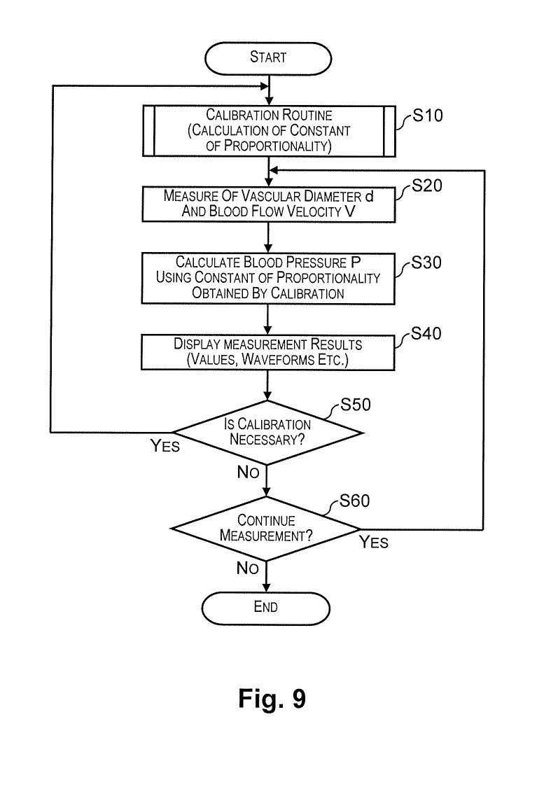

FIG. 9 is a diagram showing a blood pressure measurement method according to the present embodiment. First, the switch 37 is switched on. Then, as shown in step S10, calibration for calculation of the constant of proportionality (π/8·η·C) is performed. Details of step S10 will be described further below.

Next, as shown in step S20, the vascular diameter d and the blood flow velocity v are measured. The measurement is performed by the method described above in which the ultrasound reflection arrival time is measured and the vascular diameter d is measured, or a method in which the Doppler method is used to measure the blood flow velocity v.

Next, as shown by step S30, the constant of proportionality obtained in the calibration routine in step S10 is used to calculate the blood pressure P. The temporal change in the vascular diameter d and the blood flow velocity v at the same location and at the same time can also be obtained, and the temporal change in the blood pressure P calculated.

Next, as shown in step S40, the blood pressure P is displayed on the display part 34. The blood pressure P can also be displayed as a graph or otherwise visualized and displayed on the display part 34. The pulse may also similarly be displayed.

Next, as shown in step S50, it is determined whether recalibration is necessary. If recalibration is necessary, the flow returns to step S10 and calibration is performed. If recalibration is not necessary, the flow proceeds to step S60. An example of an instance in which calibration is necessary is an instance in which the blood pressure changes by ±15 mmHg or more compared to normal. In such an instance, a message instructing that recalibration is required is displayed on the display part 34.

Next, as shown in step S60, it is determined whether continued measurement is necessary. If continued measurement is necessary, the flow returns to step S20, and the vascular diameter d and the blood flow velocity v are measured. If continued measurement is not necessary, the flow is completed. Once a correction coefficient is initially obtained using a blood pressure value measured using a cuff-type blood pressure monitor, it is thereby subsequently possible to measure the blood pressure to a high degree of accuracy without using a cuff-type blood pressure monitor; and in an instance in which ambulatory blood pressure monitoring is performed by the subject, calibration can be performed in a simple manner without using a cuff-type blood pressure monitor.

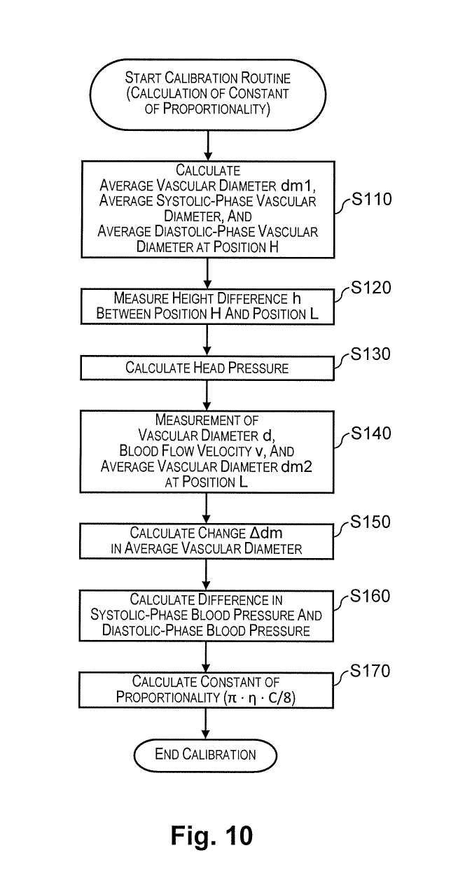

FIG. 10 is a diagram showing a calibration routine according to the present embodiment.

A flow showing the details of the calibration routine in step S10 is shown in FIG. 10. The procedure according to the head pressure conversion method (a) is as follows. First, as shown in step S110, the vascular diameter d at the position at height H in FIG. 4 is measured, and at the same time, the average vascular diameter dm1 is calculated. The change in the vascular diameter is measured for about 10 seconds.

Next, as shown in step S120, the arm is moved to the position at height L. The resulting height difference h between height H and height L is measured. A high-precision barometric pressure sensor 36 (see FIG. 3) may also be used, as a height position sensor part, to calculate the height. It is thereby possible to readily measure the height difference, which is one of the elements in terms of obtaining the head pressure.

Next, as shown in step S130, the head pressure (ρ·g·h) is calculated.

Next, as shown in step S140, the vascular diameter d and the blood flow velocity v are measured, and at the same time, the average vascular diameter dm2 is obtained.

Next, as shown in step S150, the change Δdm (=dm1−dm2) in average vascular diameter at the position at height H and height L is calculated.

Next, as shown in step S160, the pressure difference (Psys−Pdia) between the diastolic-phase blood pressure Pdia and the systolic-phase blood pressure Psys is calculated. Using the average systolic-phase vascular diameter dmsys1 and the average diastolic-phase vascular diameter dmdia1 at the position at height H in FIG. 4, Formula (21), which is a modification of Formula (9), is used to calculate the blood pressure difference (Psys−Pdia) between the diastolic-phase blood pressure Pdia and the systolic-phase blood pressure Psys.

Psys−Pdia=ρ·g·h·(dmsys1−dmdia1)/Δdm (21)

In the calculation, the blood pressure difference (Prsys−Prdia) between the actual diastolic-phase blood pressure Prdia and the actual systolic-phase blood pressure Prsys is treated as being identical to the blood pressure difference (Psys−Pdia) between the diastolic-phase blood pressure Pdia and the systolic-phase blood pressure Psys.

Next, as shown in step S170, the equation described below is used to calculate the constant of proportionality (π/8·η·C). Formula (20) is used to calculate the constant of proportionality (π/8·η·C). In the calculation, the blood pressure difference (Psys−Pdia) between the diastolic-phase blood pressure Pdia and the systolic-phase blood pressure Psys is treated as being identical to the blood pressure difference (Prsys−Prdia) between the actual diastolic-phase blood pressure Prdia and the actual systolic-phase blood pressure Prsys. The relationship between the head pressure and the change in the vascular diameter does not change. Therefore, the blood pressure difference (Psys−Pdia) between the diastolic-phase blood pressure Pdia and the systolic-phase blood pressure Psys can be calculated without cuff pressure. The constant of proportionality can thereby be readily calibrated.

According to the blood pressure measuring device and the blood pressure measuring method of the present embodiment, calibration can be performed at appropriate times in a simple manner without use of a cuff, and the blood pressure P can be measured to a high degree of accuracy. Also, it is thereby possible to provide a blood pressure measuring device and a blood pressure measuring method that are wearable and using which constant measurement is possible.

What is claimed is:

1. A blood pressure measuring device, comprising:
a blood flow velocity sensor part for transmitting/receiving a wave motion with respect to a body surface of a subject to blood within the body, and detecting a blood flow within the body;
a blood flow velocity sensor driving part for driving the blood flow velocity sensor part;
a blood flow velocity sensor signal calculating part for controlling the blood flow velocity sensor driving part and the blood flow velocity sensor part, and obtaining a blood flow velocity within the body;
a vascular diameter sensor part for transmitting/receiving ultrasound with respect to a blood vessel in the body, and detecting a difference in reflection arrival time for the vascular wall in the body;
a vascular diameter sensor driving part for driving the vascular diameter sensor part;
a vascular diameter sensor signal calculating part for controlling the vascular diameter sensor driving part and the vascular diameter sensor part, and obtaining the vascular diameter in the body; and
a blood pressure signal calculating part for using a result of a calculation by the blood flow velocity sensor signal calculating part and the vascular diameter sensor signal calculating part to obtain a blood pressure of the subject.

2. The blood pressure measuring device according to claim 1, wherein
the blood pressure signal calculating part performs a calculation to obtain the blood pressure by converting the vascular diameter to a head pressure.

3. The blood pressure measuring device according to claim 1, further comprising
a height position sensor part for obtaining a difference in the height of a predetermined portion of the subject between a first state and a second state, the first state being a state in which the predetermined portion of the subject is positioned at a predetermined height, and a second state being a state in which the predetermined portion is positioned at a height of the heart of the subject; wherein
the head pressure is obtained using the height difference measured using the height position sensor part.

4. The blood pressure measuring device according to claim 1, wherein
the blood flow velocity sensor part includes a transmitting element and a receiving element;
the transmitting element and the receiving element are provided in a plurality of pairs; and
an angle between a direction of propagation of the transmitted/received wave motion and the direction of blood flow differs between each of the pairs.

5. The blood pressure measuring device according to claim 1, wherein
the blood flow velocity sensor part is made using a piezoelectric element.

6. A method for measuring a blood pressure of a subject in a first state in which a predetermined portion of the subject is positioned at a predetermined height, the blood pressure being proportional, by a predetermined constant of proportionality, to a value obtained by dividing the blood flow velocity at the predetermined portion by the square of the vascular diameter at the predetermined portion, the blood pressure measuring method characterized in comprising:
obtaining the constant of proportionality;
measuring the vascular diameter and the blood flow velocity at the predetermined portion in the first state;
obtaining the blood pressure using the vascular diameter, the blood flow velocity, and the constant of proportionality;
displaying the blood pressure; and
determining whether calibration of the constant of proportionality is necessary.

7. The blood pressure measuring method according to claim 6, wherein the obtaining the constant of the proportionality includes
measuring, in a second state in which the predetermined portion is positioned at a height of the heart of the subject', each of the vascular diameter of the predetermined portion and the vascular diameter during a systolic phase and a diastolic phase of the predetermined portion, and obtaining a first average vascular diameter, an average systolic-phase vascular diameter, and an average diastolic-phase vascular diameter;
measuring, in the first state, a difference in the height of the predetermined portion between the first state and the second state;
using the height difference to obtain the head pressure between the first state and the second state;
measuring, in the first state, each of the vascular diameter of the predetermined portion and the blood flow velocity and the vascular diameter during the systolic phase and the diastolic phase of the predetermined portion, and obtaining a second average vascular diameter, a systolic-phase blood flow velocity, a systolic-phase vascular diameter, a diastolic-phase blood flow velocity, and a diastolic-phase vascular diameter;
using the first average vascular diameter and the second average vascular diameter to obtain a change in the average vascular diameter;
using the head pressure, the change in the average vascular diameter, the average systolic-phase vascular diameter, and the average diastolic-phase vascular diameter to obtain a blood pressure difference between a systolic-phase blood pressure and a diastolic-phase blood pressure; and
using the blood pressure difference, the systolic-phase blood flow velocity, the systolic-phase vascular diameter, the diastolic-phase blood flow velocity, and the diastolic-phase vascular diameter to obtain the constant of proportionality.

8. The blood pressure measuring method according to claim 7, wherein the measuring, in the first state, the difference in the height of the predetermined portion includes performing a measurement using a height position sensor part for measuring a difference in the height of the predetermined portion between the first state and the second state.
